(19) United States
(12) Patent Application Publication
(10) Pub. No.: US 2013/0195841 A1
(43) Pub. Date: Aug. 1, 2013

(54) BIOFILM RESISTANT MATERIALS

(71) Applicant: Jeffrey S. Gabbay, Jerusalem (IL)

(72) Inventor: Jeffrey S. Gabbay, Jerusalem (IL)

(73) Assignee: CUPRON SCIENTIFIC LTD., Jerusalem (IL)

(21) Appl. No.: 13/574,134

(22) PCT Filed: Jan. 11, 2011
(86) PCT No.: PCT/IL11/00028

§ 371 Date: Mar. 7, 2013

(30) Foreign Application Priority Data
Jan. 19, 2010 (IL) 203403

(51) Int. Cl. C08K 3/22

(52) U.S. Cl. CPC C08K 3/22
USPC 424/130.1; 523/122; 424/635

(57) ABSTRACT

This invention provides a biofilm resistant material comprising an active agent, which active agent consists essentially of an insoluble copper oxide at a concentration of between 3 and 10% w/w of said biofilm resistant material and uses thereof.

### BACKGROUND OF THE INVENTION

Bacterial biofilms exist in natural, medical, and engineering environments and pose serious threats to human health. For example, biofilms are involved in 65% of human bacterial infections. Biofilms are involved in, inter alia prostatitis, biliary tract infections, urinary tract infections, cystitis, lung infections, sinus infections, ear infections, acne, rosacea, open wounds, chronic wounds and others.

Biofilms are biological films that develop and persist at the surfaces of biotic or abiotic objects in aqueous environments from the adsorption of microbial cells into the solid surfaces. The adsorption can produce a competitive advantage for the microorganisms since they can reproduce, are accessible to a wider variety of nutrients and oxygen conditions, are not washed away, and are less sensitive to antimicrobial agents. The formation of the biofilm is also accompanied by the production of exo-polymeric materials (polysaccharides, polyuronic acids, alginates, glycoproteins, and proteins) which together with the cells form thick layers of differentiated structures separated by water-filled spaces. The resident microorganisms may be individual species of microbial cells or mixed communities of microbial cells, which may include aerobic and anaerobic bacteria, algae, protozoa, and fungi. Thus, the biofilm is a complex assembly of living microorganism embedded in an organic structure composed of one or more matrix polymers which are secreted by the resident microorganisms.

Biofilms can develop into macroscopic structures several millimeters or centimeters in thickness and over large surface areas. For non-living objects, these formations can play a role in restricting or entirely blocking flow in plumbing systems, decreasing heat transfer in heat exchangers, or causing pathogenic problems in municipal water suppliers, food process, medical devices (e.g. catheters, orthopedic devices, implants). Moreover, biofilms often decrease the life of materials through corrosive action mediated by the embedded microorganisms. This biological fouling is a serious economic problem in industrial water process systems, pulp and paper production processes, cooling water systems, injection wells for oil recovery, cooling towers, porous media (and soil), marine environments, and air conditions systems, and any closed water recirculation system. Biofilms are also a severe problem in medical science and industry causing dental plaque infections, contaminated endoscopes and contact lenses, prosthetic device colonization and biofilm formation on medical implants.

Bacteria growing in biofilms are more resistant to antibiotics and disinfectants than planktonic cells and the resistance increases with the age of the biofilm. Bacterial biofilm also exhibits increased physical resistance towards desiccation, extreme temperatures or light. As mentioned, biofilm formation causes industrial, environmental, and medical problems and the difficulties in cleaning and disinfection of bacterial biofilm with chemicals is a major concern in many industries. Furthermore, the trend towards milder disinfection and cleaning compositions may increase the insufficient cleaning of surfaces covered with biofilm.

Conventional methods of killing bacteria (such as antibiotics, and chemical disinfection) are often ineffective with biofilm bacteria. The enormous excess amount of antimicrobials required to rid systems of biofilm bacteria are undesirable and often medically impractical. Standard chemical disinfectants and antibiotics often fail to eliminate biofilms because they do not penetrate biofilms fully or fail to be fully cytocidal for species and metabolic states existing in the films. Furthermore, typical biocides kill the bacteria by damaging the cell wall structure, which in turn often results in the release of more toxic endotoxins.

A need exists for an effective redress to treat and prevent biofilm formation.

### SUMMARY OF THE INVENTION

In some embodiments, this invention provides a biofilm resistant material comprising an active agent, which active agent consists essentially of an insoluble copper oxide at a concentration of between 3 and 10% w/w of said biofilm resistant material.

In some embodiments, this invention provides a method of inhibiting, diminishing or abrogating microbial biofilms or microbial biofilm formation or a combination thereof on a material surface, said method comprising producing said material with at least a surface comprising an active agent consisting essentially of an insoluble copper oxide at a concentration of between 3 and 10% w/w of said biofilm resistant material.

In some embodiments, this invention provides a method of inhibiting, diminishing or abrogating microbial biofilms or microbial biofilm formation or a combination thereof on a material surface, said method comprising attaching to a portion of a surface of said material an active agent consisting essentially of an insoluble copper oxide such that said insoluble copper oxide is at a concentration of between 3 and 10% w/w of said material.

### BRIEF DESCRIPTION OF THE DRAWINGS

FIG. 1 graphically depicts copper bacteriocidal activity.

FIG. 2 graphically depicts copper bacteriocidal activity upon repeat exposure.

### DETAILED DESCRIPTION OF THE PRESENT INVENTION

This invention provides, inter alia, biofilm resistant materials comprising an active agent, which active agent consists essentially of an insoluble copper oxide at a concentration of between 3 and 10% w/w of said biofilm resistant material.

In some embodiments, the biofilm resistant material, further comprises a polymeric resin.

In some embodiments, the polymeric resin is a polypropylene, polycarbonate, polyurethane, polyvinyl chloride, nylon, polystyrene, polyethylene, polyethylene terephthalate, fluorinated polyethylene, polyvinyl alcohol, polyvinyl acetate, silicone or polyester.

According to this aspect, and in one embodiment, the polymer is a block copolymer, or in another embodiment, the polymer is a polysaccharide, which in some embodiments is a poly(pyranose) or a poly(furanose) or a combination thereof, or in other embodiments, is a dextran or an inulin.

In some embodiments, the polymer may comprise, inter-alia, poly(pyranose), poly(hydroxyl acid), poly(lactone), poly(amino acid), poly(anhydride), poly(methane), poly(orthoester), poly(phosphazine), poly(phosphoester) or poly(lactic-co-glycolic) acid, poly(ether ester)s, synthetic poly(amino acids), polycarbonates, poly(hydroxyalkanoate)s, and poly(caprolactone)s

In one embodiment, the polymer is a synthetic polymer, or in another embodiment, the polymer is a natural polymer. In one embodiment, the polymer is a poly(cianoacrylate), poly(alkyl-cianoacrylate), poly(ketal), poly(caprolactone), poly(acetal), poly(hydroxy-ester), poly(hydroxyl-alkanoate), polypropylene-fumarate), poly poly(ester), poly(ethers), poly(carbonates), poly(amide), poly(siloxane), poly(silane), poly(sulfide), poly(imides), poly(urea), poly(amide-enamine), poly(organic acid), poly(electrolytes), poly(p-dioxanone), poly(olefin), poloxamer, inorganic or organometallic polymers, elastomer, or any of their derivatives, or a copolymer obtained by a combination thereof

In one embodiment, the polymer comprises poly(D,L-lactide-co-glycolide) (PLGA) In another embodiment, the polymer comprises poly(D,L-lactide) (PLA). In another embodiment, the polymer comprises poly(D,L-glycolide) (PGA) or poly(glycerol sebacate), PGSA. In one embodiment, the polymer comprises a glycosaminoglycan

In one embodiment, the polymer may comprise proteins such as zein, modified zein, casein, gelatin, gluten, serum albumin, collagen, actin, α-fetoprotein, globulin, macroglobulin, cohesin, laminin, fibronectin, fibrinogen, osteocalcin, osteopontin, osteoprotegerin, or others, as will be appreciated by one skilled in the art In another embodiment the polymer may comprise cyclic sugars, cyclodextrins, synthetic derivatives of cyclodextrins, glycolipids, glycosaminoglycans, oligosaccharides, polysaccharides such as alginate, carageenan, chitosan, celluloses, chondroitin sulfate, curdlan, dextrans, elsinan, fuicellran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluronic acid, chitin, poly(3-hydioxyalkanoate)s, such as poly(hydroxybutyrate), poly(3-hydtoxyoctanoate) or poly(3-hydroxyfatty acids) In another embodiment, the polymer may comprise chemical derivatives thereof (substitutions, additions, and elimination of chemical groups, for example, alkyl, alkylene, hydroxylation, oxidation, and other modifications routinely made by those skilled in the art), blends of, e g proteins 01 carbohydrates alone or in combination with synthetic polymers.

In one embodiment, the polymer comprises synthetically modified natural polymers, and may include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxyropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt

In one embodiment, the polymer comprises synthetic degradable polymers, which may include, but are not limited to polyhydroxy acids, such as poly(lactide)s, poly(glycolide)s and copolymers thereof; poly(ethylene terephthalate); poly(hydroxybuty tic acid); poly(hydroxyvaleric acid); poly(pseudo amino acids); poly(amino acids); poly(hydroxyalkanoate)s; poly(anhydrides); poly(orthoester)s; and blends and copolymers thereof

In one embodiment, the polymer comprises a bioerodible polymer such as poly(lactide-co-glycolide)s, poly(anhydride)s, and poly(orthoester)s, which have carboxylic groups exposed on the external surface as the smooth surface of the polymer erodes, which may also be used In one embodiment, the polymer contains labile bonds, such as polyesters.

In some embodiments, the biofilm resistant material comprises a second active ingredient which is an effector compound and which second active ingredient is not a bioactive metal.

In some embodiments, the effector compound is an anti-biotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, a growth factor, a hormone, a DNA, an siRNA, a vector or any combination thereof. In some embodiments, the antifungal is a polyene antifungal. In some embodiments, the antifungal is Amphotericin.

In one embodiment, the polymer is biodegradable. In one embodiment, the term “biodegradable polymer”' refers to a material, which is degraded in the biological environment of the cell or subject in which it is found In one embodiment, the biodegradable polymer undergoes degradation, dining which, acidic products, or in another embodiment, basic products ate released In one embodiment, bio-degradation involves the degradation of the polymer into its component subunits, via, for example, digestion, by a biochemical process In one embodiment, biodegradation may involve cleavage of bonds (whether covalent or otherwise) in the polymer backbone In another embodiment, biodegradation may involve cleavage of a bond (whether covalent or otherwise) internal to a side-chain or one that connects a side chain to the polymer backbone

In one embodiment, this invention provides a coated material as described herein, wherein the substrate is a particle, which is of any size which finds application in the methods as described herein, in some embodiments In some embodiments, the particle is of a diameter ranging from about 1-900 nanometer, or in another embodiment, the particle is of a diameter ranging from about 1-900 micrometer, or in another embodiment, the particle is of a diameter ranging from about 0.1-10 millimeters

In one embodiment, the In some embodiments, the material comprises a rubber, a ceramic, a composite stone or a marble.

In some embodiments, the biofilm resistant material comprising an active agent, which active agent consists essentially of an insoluble copper oxide is characterized in that the insoluble copper oxide is a part of the composition of the material, and in some embodiments, the material is characterized in that the material further comprises a coating associated therewith, in which a component of the coating is insoluble copper oxide, and which coating then comprises insoluble copper oxide at a concentration of between 3 and 10% w/w.

In one embodiment, the material is a part of, or in the form of a bead, microparticle, nanoparticle, sponge, bandage, suture, catheter, stent, valve, pacemaker, conduit, cannula, appliance, scaffold, contraceptive device, central line, pessary, tube, drain, trochar or plug, In one embodiment, the catheter is a PA, pericardial, pleural, urinary or intra-abdominal catheter.

In one embodiment, the drain is a cerebrospinal fluid drain

In one embodiment, the tube is a tracheostomy, endotracheal or chest tube

In another embodiment, the substrate is a part of, or in the form of an implant, a rod, a screw, or an orthopedic appliance. In another embodiment, the substrate is a part of, or in the form of a pipe lining, equipment which comes into contact with food or seawater or wastewater

In one embodiment, the substrate is a part of, or in the form of a bandage. In one embodiment, the substrate is a part of, or in the form of a suture.

In one embodiment, the substrate is a part of, or in the form of a catheter. In one embodiment, the catheter is a PA, pericardial, pleural, urinary or intra-abdominal catheter In another embodiment, the catheter is a coronary catheter, epidural catheters peripheral vascular catheter, or neuro-interventional microcatheter

In one embodiment, the substrate is a part of, or in the form of a stent. In one embodiment, the stent may comprise, inter-alia, an endovascular, biliary, tracheal, gastrointestinal, urethral, ureteral, esophageal and/or coronary stent. In one embodiment, the stent may comprise, inter-alia, a stent in the airway, hepatobiliary tract, and others, as will be appreciated by one skilled in the art

In another embodiment, the biofilm resistant material is a part of, or in the form of an embolic coil, endovascular graft, guide wire, stylets, introduces, and/or balloon, and the like. In one embodiment, the balloon may comprise, inter-alia, a coronary balloon, peripheral vascular balloon, and/or neurological balloon.

In some embodiments, the coated stents include, for example, vascular stents such as self-expanding stents and balloon expandable stents. Examples of self-expanding stents useful in the present invention, and representing embodiments thereof, are in U.S. Pat Nos. 4,655,771; 4,954,126; 5,061,275. In some embodiments, the stent which may be coated and comprise embodiments of the invention, or are for use according to the methods of this invention include, for example, an express stent such as the Express I M stent.

The materials of this invention may comprise metallic, ceramic, or polymeric materials, or a combination thereof.

In some embodiments, the metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

In some embodiments, the ceramic materials include, but are not limited to. oxides, carbides, or nitrides of the transition elements such as titanium oxides, hafnium oxides, iridium oxides, chromium oxides, aluminum oxides, and zirconium oxides Silicon based materials, such as silica, may also be used

In some embodiments, the coating of a material will provide characteristics to the material appropriate to the particular application of use.

In one embodiment, the material is a part of, or in the form of a valve. In one embodiment, the substrate is a part of, or in the form of a pacemaker. In one embodiment, the substrate is a part of, or in the form of a conduit. In one embodiment, the substrate is a part of, or in the form of a cannula.

The terms “substrate” and “material” are used interchangeably herein and refer to the structure which incorporates the active ingredient consisting essentially of copper oxide, as herein describe.

In one embodiment, the substrate is a part of, or in the form of an appliance.

In one embodiment, the substrate is a part of, or in the form of a tissue scaffold.

In one embodiment, the substrate is a part of, or in the form of a central line.

In one embodiment, the substrate is a part of, or in the form of a pessary

In one embodiment, the substrate is a part of, or in the form of a tube. In one embodiment, the tube is a tracheostomy, gastostomy tube, F-tube, enteral feeding device, endotracheal or chest tube

In one embodiment, the substrate is a part of, or in the form of a drain. In one embodiment, the substrate is a part of, or in the form of a trochar or plug. In one embodiment, the drain is a cerebrospinal fluid drain.

In another embodiment, the substrate is a part of, or in the form of an implant. In one embodiment, the substrate is a part of, or in the form of a rod. In one embodiment, the substrate is a part of, or in the form of a screw. In one embodiment, the substrate is a part of, or in the form of an orthopedic appliance

In another embodiment, the invention provides for coated implants, which may include, but are not limited to, vascular grafts, soft and hard tissue prostheses including, but not limited to, pumps, electrical devices including stimulators and recorders, auditory prostheses, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves

In another embodiment, the substrate is a part of, or in the form of a contraceptive device. In one embodiment, the substrate may be a part of, or in the form of, a diaphragm, a condom, a cervical cap, and the like.

In another embodiment the substrate is a part of, or in the form of a product used for feminine hygiene. In one embodiment, such a product may include, inter-alia, a tampon, a padding, including sanitary napkin padding or nursing padding.

In another embodiment, the materials of this invention may comprise, be in the form of, or be a part of tracheal devices, such as endo-tracheal tubes, aspirating devices and other tracheal suction devices, broncho-alveolar lavage catheters

This invention provides, in some embodiments, the materials come into contact with human tissue. According to this aspect, and in one embodiment, the material may be a surgical material, such as, surgical instruments, suture material, implantable material, etc.

In some embodiments, such materials for which coating is envisaged will include, inter-alia, surgical, medical or dental instruments, bandages, patches, prosthesis, appliances, implants, scaffolding, suturing material, valves, pacemaker, stents, catheters, rods, shunt, tubing, wiring, electrodes, clips or fasteners, monitors, e g., fetal monitors, contraceptive devices, feminine hygiene products, casting, endoscopes s and any others, which come into contact with human tissue.

In another embodiment, the substrate is a part of, or in the form of a pipe lining, or equipment which comes into contact with food or seawater or wastewater

It is to be understood that any material, used for any purpose, which incorporates as active ingredient an insoluble copper oxide as herein described, is to be considered an embodiment of this invention.

In one embodiment, the compound is released slowly, over a course of time, or in another embodiment, the compound is minimally released over a course of time.

In one embodiment, the material may be affixed, glued, or sutured to the skin, or pierce the skin, or in another embodiment, the material serves as a portal through which other materials are passed through the skin.

In some embodiments, this invention provides a versatile platform for creating substrates, particles, rods, spheres, devices, etc, on or within which, insoluble copper oxide is incorporated, which prevents or treats biofilm formation thereupon.

As described herein, the invention also contemplates biofilm resistant materials incorporating a coating, which coating comprises an active ingredient, which active agent consists essentially of an insoluble copper oxide at a concentration of between 3 and 10% w/w of the biofilm resistant materials.

In one embodiment the term “coated” refers to the physical attachment, or, in another embodiment, association of coating comprising the insoluble copper oxide as herein described with at least a portion of a surface of a material whose “coating” is desired In one embodiment, such coating will comprise less than 1% of an exposed surface of the material, or in another embodiment, from 1-10%, or in another embodiment, from 1-25%, or in another embodiment, from 1-50%, or in another embodiment, from 1-75%, or in another embodiment, from 1-100% of at least one surface of the material

In one embodiment, application of such “coating” will be in a pattern, or on specific regions of the material to suit a particular purpose For example, and in some embodiments, tubing may comprise coating of one material on the luminally exposed surface of the tube.

The coating applied to the materials of this invention may comprise films, particles etc.

A material for application in a subject with a cardiovascular disease or condition may be administered a coated material, such as a stent or balloon catheter, which may comprise the active agent, incorporated within the stent or balloon catheter, or in some embodiments, a coating comprising the active agent, on the stent or balloon catheter.

In one embodiment, the coating of a material will be on at least one surface of the material, or in another embodiment, on two or more surfaces of the material, or in another embodiment, on every exposed surface of the material, or in another embodiment, on any surface of the material

In one embodiment, the term “coated material” applies not only to a surface coating of the material, but is to be understood as encompassing embedding and/or impregnating the material, in whole, or in some embodiments, in part, with the coatings described herein, or in some embodiments, the embedding and/or impregnating the material may be according to a desired pattern and/or design, to suit a particular purpose or application. In some embodiments, multiple coatings may be impregnated or embedded in the material, each of which may be applied according to a particular pattern or design, which may be the same, or in another embodiment, different than the patterning of a first coating, for example, varying in terms of the percent by weight of the insoluble copper oxide, or in some embodiments, in terms of the composition of the coating, irrespective of the copper oxide component.

In some embodiments, the embedding and/or impregnating of the material may be to a particular surface of a material, in a particular pattern and/or design, to suit a particular purpose or application In some embodiments, the embedding and/or impregnating of the material may be to two or more surfaces of the material in the particular pattern and/or design, or such pattern and/or design may vary as a function of the surface to which the material is being embedded and/or impregnated within.

In some embodiments, the invention particularly relates to any sponge-like material which can be polyurethane or other material made into such form.

In some embodiments, the materials of this invention will make use of different polymers, which in turn will require variations in the formulation and preparation of the copper oxide for its inclusion in the polymer carrier, as will be appreciated by the skilled artisan. Polymers such as polyester (PET) and polyaramide (PA) require separation of the copper oxide for effective incorporation within structures containing such polymers.

The skilled artisan will also appreciate which polymers will not react with the copper oxide, so as to allow appropriate incorporation of the insoluble copper oxide therein.

For example, for materials such as PET, PA, PP, PE, etc. the materials may be prepared by isolating the copper oxide during the extrusion process followed by the addition of a dispersing agent. Materials such as PU or latex may incorporate insoluble copper oxide via encapsulation of copper oxide powder therewithin, and materials such as silicone may incorporate insoluble copper oxide by mixing the silicone with powdered copper oxide.

It is to be understood that the preparation of the polymers may be by any means known in the art. For example, and in some embodiments, a polymer concentrate comprising at least one of an oxide of copper, a polyethylene wax prepared from ethylene and, if desired, one or more olefins using a chelating agent catalyst, if desired a thermoplastic polymer and, if desired, other additives is prepared and added to a carrier polymer in the form of a master batch concentration. In some embodiments, only a partial preparation of the powder is necessary, such as only dispersing the powder in a wax, and in some cases the powder can be added directly to the carrier.

In some embodiments, a master batch formulation is prepared at a concentration of between 20% and 40% copper oxide w/w for inclusion in the slurry state of a polymer for use. The master batch may be diluted to between 3% and 10% copper oxide to achieve the desired composition.

A typical formulation would comprise, in some embodiments, in addition to the copper oxide, a dispersant, for example a wax, and, if desired, a thermoplastic polymer, usually a polyolefin. The thermoplastic polymer assists the dispersing of the wax throughout the slurry. The wax serves to disperse the copper oxide and other additives evenly and finely in the formulation and to stabilize this dispersion. In some embodiments, an envisioned formulation of such a concentrate for a master-batch would comprises 20-40% by weight of copper oxide, 10% by weight of wax, 2% chelating agent and the balance by weight of a thermoplastic polymer. The ratios of the individual components can be varied within certain limits.

In some embodiments, a thermoplastic polymer is utilized as a carrier polymer; this polymer is, for example, polyester, polyamide, polyethylene, polypropylene, polystyrene, polyoxymethylene, a polystyrene copolymer such as a styrene-butadiene copolymer, an acrylonitrile-butadiene-styrene terpolymer. In some embodiments, an antioxidant is added from 0 to 10% by weight if there are impurities in the copper oxide that will upset the organic carrier. The ratios of the various components can be varied within wide limits. The ratios are matched so that they add up to a total of 100% by weight. In some embodiments, chemical pretreatment of the support material is also possible.

Suitable polymers may include, in some embodiments,: polyethylene, polypropylene, polyester, polystyrene, polyoxymethylene, polyethylene terephthalate, polybutylene terephthalate, polymethyl methacrylate, polyether sulfones, polysulfones, polyether ketones, polystyrene copolymers, acrylonitrile-butadiene-styrene terpolymers, polyamides such as nylon 6 or nylon 6.6, polyvinyl chloride and copolymers of ethylene but certainly not limited only to these which are the most common. Polymers such as PGA or other dissolvable polymers can also be formulated using the above method.

Incorporation of the insoluble copper oxide within the materials as herein described may make use of a chelating agent, as will be appreciated by the skilled artisan. Chelating agents that can be used in the present invention, may include such compounds as: Diethylenetriaminepentaacetic acid (dtpa), Ethylenedinitrilotetraacetic acid (edta), Nitrilotriacetic acid (nta), Ethylenediamine (eda), Diethyltriamene (deta), Triethylenetetraamine (teta), Tetraethylenepentamine-UHP (tepa-UHP), Pentaethylenehexamine (peha), Piperazine, and mixtures thereof.

The present invention relates to methods for preventing, removing, or reducing biofilm on a surface, comprising contacting the surface with an effective amount of a composition comprising one or more cationic species of copper oxide. The methods of the present invention may be used to prevent, remove, reduce, or disrupt biofilm formation on a surface. One of ordinary skill in the art will recognize that using copper oxide as the active ingredient to attack the biofilm has application to a variety of hard surfaces. The copper oxide can be included in a polymer, or attached to a polymer after it is shaped, can be placed as a coating on a hard surface, or included in a polymer that is then attached to a hard surface, or can be applied directly to the materials from which the surface material is constructed such as rubber (artificial and natural), urethane compounds, polymers, ceramics, composite stones or marbles, products used in the formation of table tops, and softer materials such as those used in floorings.

This invention provides, in other embodiments, for a method of inhibiting, diminishing or abrogating microbial biofilms or microbial biofilm formation or a combination thereof on a material surface, the method comprising producing such material with at least a surface comprising an active agent consisting essentially of an insoluble copper oxide at a concentration of between 3 and 10% w/w of the biofilm resistant material.

This invention provides, in other embodiments, for a method of inhibiting, diminishing or abrogating microbial biofilms or microbial biofilm formation or a combination thereof on a material surface, said method comprising attaching to a portion of a surface of said material an active agent consisting essentially of an insoluble copper oxide such that said insoluble copper oxide is at a concentration of between 3 and 10% w/w of said material.

### EXAMPLES

### Example 1

### Bacteriocidal Effects of Copper

### Materials and Methods:

### Primary Exposure:

Polypropylene plates with and without a hydrophilic additive served as controls. Where a hydrophilic additive was used the commercially available surfactant/lubricant Lurol in a 0.5% to 2% concentration was used. Polypropylene plates containing 5% Copper oxide (weight/weight) with and without the hydrophilic additive were prepared and 100 μl of saline containing approximately 10⁵ CFU Escherichia coli ATCC #8739 were put on top of the plates.

### Multiple Repetitive Exposures:

One hundred μl saline containing approximately 10⁵ CFU bacteria were put on top of polypropylene plates (same plates as detailed above) and incubated in a moist chamber at 37° C. After 1 hour the 100 μl saline aliquot was removed and a fresh 100 μl aliquot of saline containing approximately 10⁵ CFU bacteria were added on top of the polypropylene plates exactly at the same spot where the previous saline aliquot was placed. This was repeated 3 more time after 3, 18 and 42 hours from the beginning of the experiment. After the final spiking of the polypropylene plates, the plates were kept in a moist chamber at 37° C. before the final aliquots were removed and the number of CFU in each aliquot was determined.

### Results

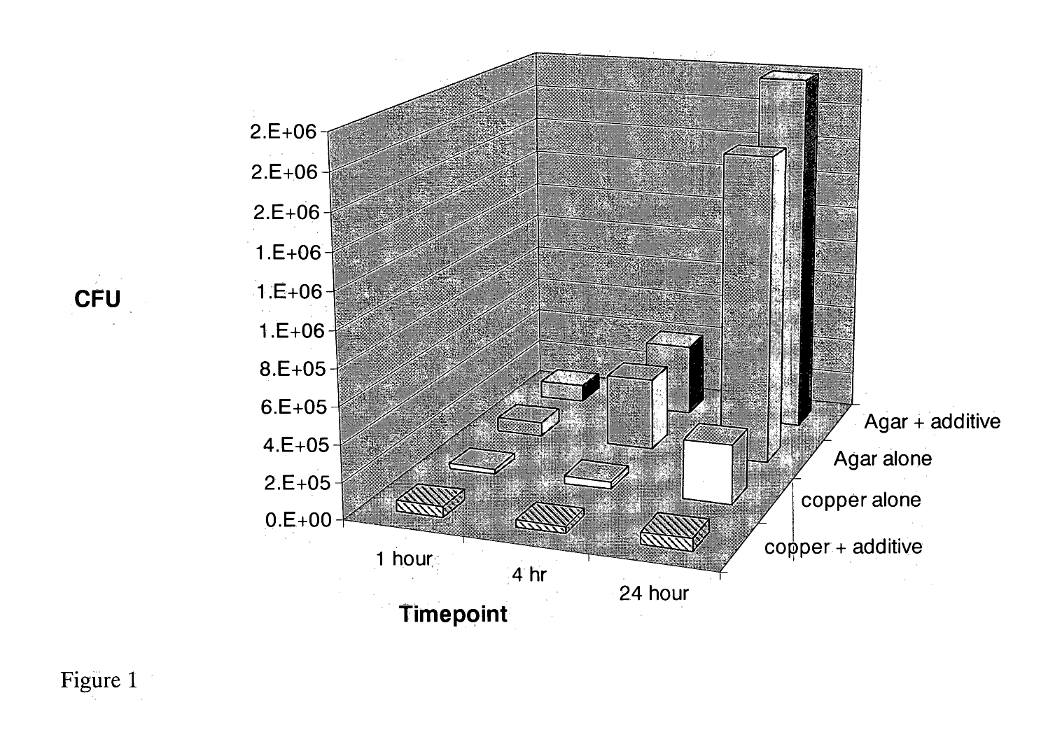

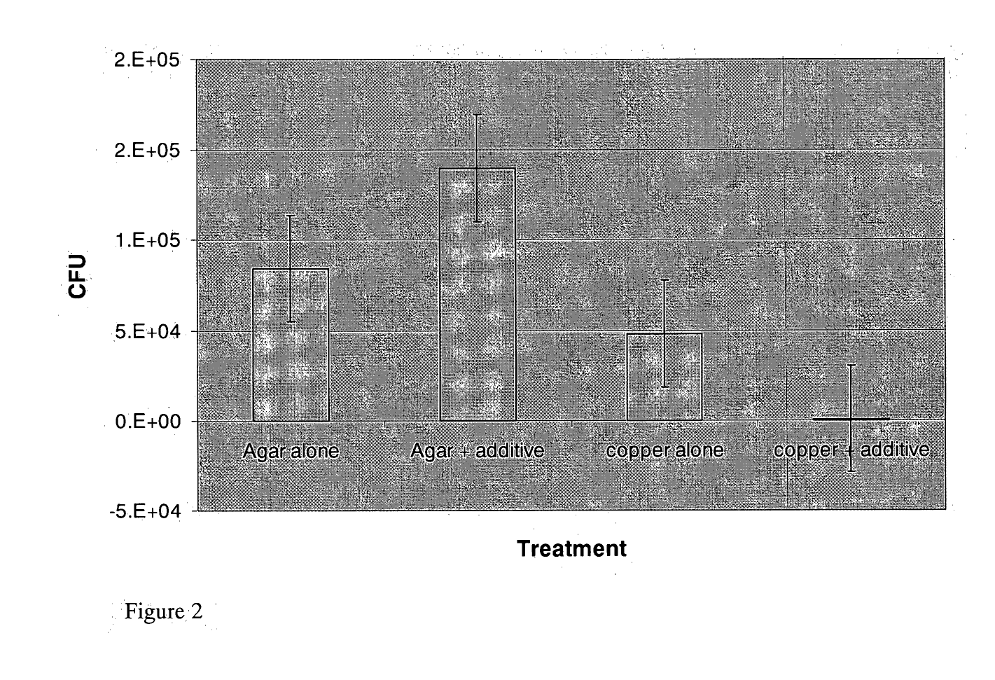

In order to test the bactericidal effect of copper on bacterial growth, E. coli was exposed to polypropylene plates containing 5% copper oxide (weight/weight) and FIG. 1 depicts the effect of copper on bacterial growth. FIG. 2 depicts the effect of cooper on bacterial growth for a substrate with a repeat exposure proving that the substrate is efficacious for a prolonged period of time and for sequential bacterial exposures. In both cases, the CFU were reduced in plates containing copper oxide.

### Example 2

### Effects of Copper on Biofilm Formation

In order to determine the effect of insoluble copper containing formulations on the presence or absence of a biofilm, the following assay was conducted: Polypropylene plates 0.4 cm×0.4 cm in size (with and without 5% Cu (w/w)) in duplicates were placed at the bottom of 24 disposable well plates. 1 ml of Escherichia coli ATCC #8739 (˜10⁵ colony forming units) were added to the wells and incubated overnight at 37° C. (approximately 10 hours). After the incubation, the plates were fixed with glutaraldehyde 5% (in phosphate buffer 0.1M PH 7.2) for 2 hrs, rinsed with phosphate buffer 4 times (10 minutes each wash) and dehydrated with a solution containing an increasing ethanol concentration. Samples were dried by a Critical Point Dryer (BIO-RAD C.P.D 750), mounted on metal stubs, sputter coated with gold and evaluated by scanning electron microscopy (Jeol JMS 840). The presence or absence of a biofilm was visually ascertained and recorded following scanning multiple fields.

Samples assessed contained 0% Cu (w/w) plates (control 1), or 0% Cu (w/w) plates containing a hydrophilic additive as described in Example 1 (control 2), 5% Cu (w/w) plates (Cul) or 4. 5% Cu (w/w) plates with the hydrophilic additive (Cu2)

Table 1 provides the results of the SEM studies:

TABLE 1
Biofilm formation following treatment with insoluble copper:

| Control 1 | Control 2 | Cu 1 No | Cu 2 No |
|---|---|---|---|
| + | + | − | − |

+ represents formation of a biofilm, while − represents the absence of a biofilm.

The results of Table 1 indicate that E. coli grown on plates containing insoluble copper failed to form a biofilm and therefore the insoluble copper substrate prevented biofilm formation.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A biofilm resistant material comprising an active agent, which active agent consists essentially of an insoluble copper oxide at a concentration of between 3 and 10% w/w of said biofilm resistant material.

2. The biofilm resistant material of claim 1, further comprising a polymeric resin.

3. The biofilm resistant material of claim 1, wherein said polymeric resin is a polypropylene, polycarbonate, polyurethane, polyvinyl chloride, nylon, polystyrene, polyethylene, polyethylene terephthalate, fluorinated polyethylene, polyvinyl alcohol, polyvinyl acetate, silicone or polyester.

4. The biofilm resistant material of claim 1, wherein said material is a sponge, a catheter, a pipe, a lining, a tube, a bandage, a suture, a stent, a valve, a pacemaker, a conduit, a cannula, an appliance, a scaffold, a central line, a pessary, a drain, a trochar or a plug.

5. The biofilm resistant material of claim 4, wherein said catheter is a PA, pericardial, pleural, urinary or intra-abdominal catheter.

6. The biofilm resistant material of claim 4, wherein said drain is a cerebrospinal fluid drain

7. The biofilm resistant material of claim 4, wherein said tube is a tracheostomy, endotracheal or chest tube.

8. The biofilm resistant material of claim 1, wherein said material is a part of, or in the form of an implant, a rod, a screw, or an orthopedic appliance.

9. The biofilm resistant material of claim 1, wherein said material is a part of, or in the form of a pipe lining, a reactor, or equipment which comes into contact with food or seawater

10. The biofilm resistant material of claim 1, wherein said material is a part of, or in the form of a receptacle, reservoir, or container.

11. The biofilm resistant material of claim 1, wherein said insoluble copper oxide is released slowly, over a course of time.

12. The biofilm resistant material of claim 1, wherein said material may be affixed, glued, or sutured to the skin, or pierce the skin.

13. The biofilm resistant material of claim 1, wherein said material serves as a portal through which other materials are passed through the skin

14. The biofilm resistant material of claim 1, wherein said material comprises a second active ingredient which is an effector compound and which second active ingredient is not a bioactive metal.

15. The biofilm resistant material of claim 14, wherein said effector compound is an anti-biotic, an antiviral, an antifungal, an anti-helminth, an anti-inflammatory, an antihistamine, an immunomodulatory, an anticoagulant, a surfactant, a bronchodilator, an antibody, a beta-adrenergic receptor inhibitor, a calcium channel blocker, an ace inhibitor, a growth factor, a hormone, a DNA, an siRNA, a vector or any combination thereof.

16. The biofilm resistant material of claim 14, wherein said antifungal is a polyene antifungal.

17. The biofilm resistant material of claim 14, wherein said antifungal is Amphotericin.

18. A method of preventing, diminishing or reducing the incidence of microbial biofilm formation on a material surface, said method comprising producing said material with at least a surface comprising an active agent consisting essentially of an insoluble copper oxide at a concentration of between 3 and 10% w/w of said biofilm resistant material.

19. A method of preventing, diminishing or reducing the incidence of microbial biofilm formation on a material surface, said method comprising attaching to a portion of a surface of said material an active agent consisting essentially of an insoluble copper oxide such that said insoluble copper oxide is at a concentration of between 3 and 10% w/w of said material.
